(19) United States
(12) Plant Patent Application Publication
(10) Pub. No.: US 2022/0248582 P1
(43) Pub. Date: Aug. 4, 2022

(54) Blueberry plant named C15-270

(50) ;

(71) Applicants: Costa Berry International Pty Ltd, Ravenhall (AU); FLORIDA FOUNDATION SEED PRODUCERS, INC., Marianna, FL (US)

(72) Inventors: Jessica Scalzo, Corindi Beach (AU); James W. Olmstead, Aptos, CA (US)

(21) Appl. No.: 17/803,047

(22) Filed: Jan. 26, 2022

Related U.S. Application Data
(60) Provisional application No. 63/206,157, filed on Jan. 29, 2021.

(51) Int. Cl. A01H 6/36

(52) U.S. Cl. PLT/157

(57) ABSTRACT

The new blueberry plant variety ‘C15-270’ is provided. ‘C15-270’ is a commercial variety intended for the fresh market. The variety is produced from a cross of unpatented parents ‘FL12-082’ and ‘FL12-069’.

### CROSS-REFERENCE TO RELATED APPLICATIONS

This application claims priority to U.S. Application No. 63/206157 filed on 29 Jan. 2021, the contents of which are hereby incorporated in their entirety by reference.

Latin name of the family, genus, and species: Family—Ericaceae. Genus—Vaccinium. Species—corymbosum hybrid.

Variety denomination: The new blueberry plant claimed is of the variety denominated ‘C15-270’.

### BACKGROUND OF THE INVENTION

The new variety ‘C15-270’ was selected from a population of seedlings derived from crossing the blueberry varieties known as ‘FL12-082’ (unpatented seed parent) and the variety known as ‘FL12-069’ (unpatented pollen parent). The cross was made in 2012 in Florida, USA and the seed was sown and grown in Corindi Beach, New South Wales, Australia. The new variety was selected in 2015 from among plants located on land at Corindi Beach and assigned the breeding code ‘C15-270’. Plants of ‘C15-270’ were propagated by cuttings for further evaluation and resulted to be uniform and stable. The new variety showed distinctive traits such as evergreen, with fruit of good flavor, large fruit size, and firmness.

### SUMMARY OF THE INVENTION

The new variety ‘C15-270’ was originated from a cross of ‘FL12-082’ (unpatented seed parent) and the variety known as ‘FL12-069’ (unpatented pollen parent) in 2012 in Florida, USA.

The new blueberry variety resulted from seedlings produced in a controlled breeding programme. The cross was made in 2012 in Florida, USA and the seed was sown and grown on in Corindi Beach, New South Wales, Australia.

The new variety was selected in 2015 from among plants located on land at Corindi Beach and has since been named ‘C15-270’. Since then plants of ‘C15-270’ were propagated by cuttings for further evaluation and confirmed to be uniform and stable. Asexual reproduction of the new variety ‘C15-270’ by cutting propagation since 2015 at Corindi Beach, New South Wales, Australia has demonstrated that the new variety reproduces true to type plants.

The new variety was selected in 2015 as a single plant within a population of seedlings resulting from controlled cross of Vaccinium varieties. The seedling population was planted in an experimental block in the field at Corindi Beach, New South Wales, Australia and the selection of the new variety took place in the same block. Selection criteria were a combination of late cropping season, low chilling requirement, strong plant vigour, non-deciduous type of plant (evergreen), large fruit size, good fruit flavor, and very firm fruit. The new variety was subsequently evaluated for five years at the commercial farm at Corindi Beach, New South Wales, Australia.

The following characteristics of the new variety have been repeatedly observed and can be used to distinguish ‘C15-270’ as a new and distinct variety of Vaccinium corymbosum hybrid:
1. Non-deciduous (Evergreen)
2. Late season crop
3. Strong plant vigour
4. High yields
5. Low chilling requirement
6. Excellent firmness and good flavor

The new blueberry variety ‘C15-270’ has maintained its distinguished characteristics throughout successive asexual propagation. The variety has been repeatedly asexually reproduced through softwood cuttings in New South Wales, Australia and the clones are phenotypically identical to the original plant.

### BRIEF DESCRIPTION OF THE DRAWINGS

The accompanying photographic illustration shows typical specimens in full color of the foliage and fruit of the new variety ‘C15-270’. The colors are as nearly true as is reasonably possible in a color representation of this type.

FIG. 1 is a photograph of three-year-old plants of the new variety ‘C15-270’.

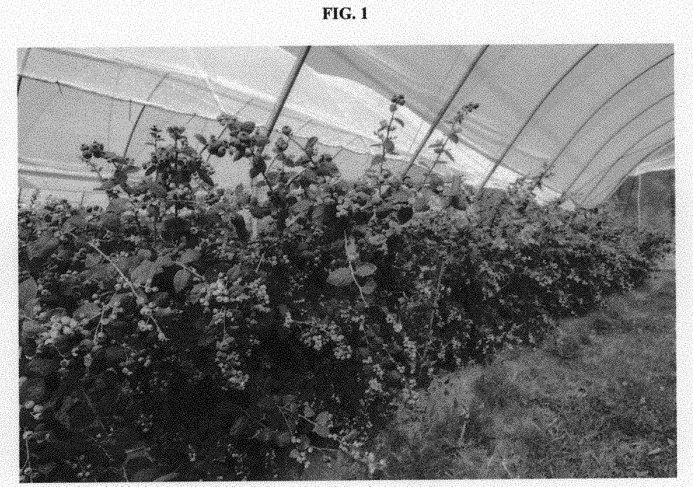

FIG. 2 is a photograph of the typical fruit cluster of the new variety ‘C15-270’.

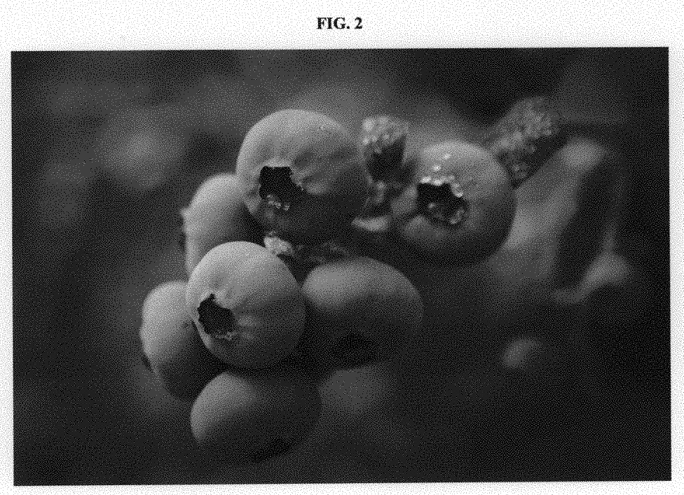

FIG. 3 is a photograph of the botanical parts (ripe fruit, green fruit, flowers and leaves) of the new variety ‘C15-270’.

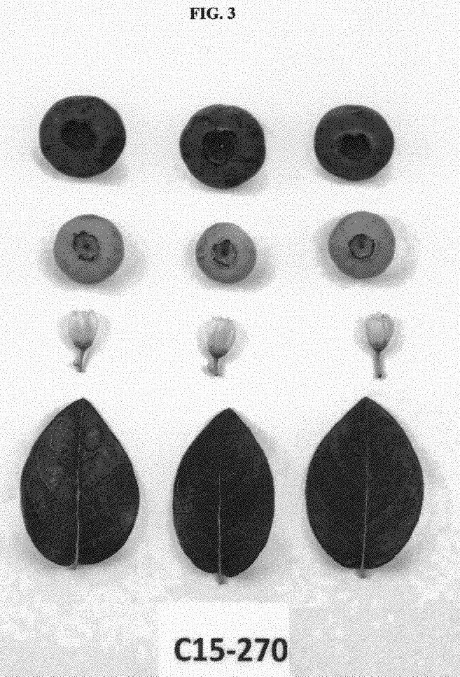

FIG. 4 is a photograph showing the medium to large pedicel scar of the new variety ‘C15-270’.

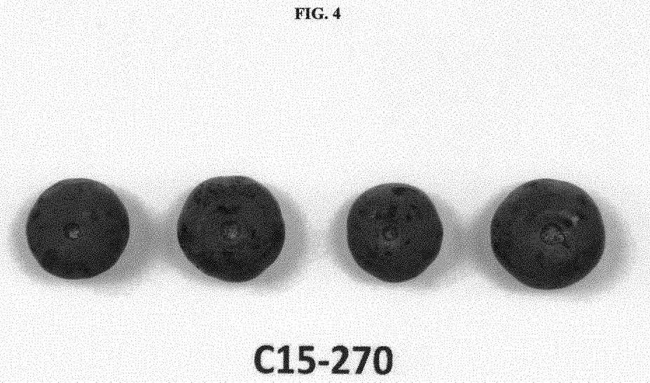

The colors in the photographs are as close as possible with the photographic and printing technology utilized. The color values cited in the detailed botanical description accurately describe the colors of the new blueberry variety.

### DETAILED BOTANICAL DESCRIPTION

The following detailed description sets forth the distinctive characteristics of ‘C15-270’. The data which defines these characteristics was collected from asexual reproductions of the original selection. Dimensions, sizes, colors, and other characteristics are approximations and averages set forth as accurately as possible. For all traits data was collected from 3 plant parts across 6 randomly selected plants. For the traits relating to fruits (e.g., fruit weight, firmness, brix, acidity) the data is an average across twenty fruits collected randomly. The plant history was taken on plants approximately 3 years of age, and the descriptions relate to plants grown in the field in Corindi Beach, New South Wales, 2456 Australia. Descriptions of fruit characteristics were made on fruit grown in Corindi Beach, New South Wales, 2456 Australia. Color designations are from the 2007 edition of The Royal Horticultural Society (“R.H.S.”).
Classification:
   a. Family.—Ericaceae.
   b. Genus.—Vaccinium.
   c. Species.—Corymbosum hybrid.
   d. Common name.—Blueberry.
Parentage: Female Parent—unpatented selection ‘FL12-082’. Male Parent—unpatented selection ‘FL12-069’.
Market class: fresh market.
Plant:
General:
   Parentage.—FL12-082×FL12-069.
   Plant height.—1.45 m.
   Plant width.—1.26 m.
   Growth habit.—Semi-upright.
   Growth.—Strong vigour.
   Productivity.—High yield.
   Cold Hardiness.—Low chill.
   Cold tolerance.—Low.
   Chilling requirement.—Low, estimated between 150 and 300 hours.
   Tolerance to disease.—Tolerance to leaf rust.
   Leafing.—Overall strong leafing, and plant retains leaves during the winter.
   Twigginess.—Low.
Foliage:
General:
   Leaf color (top side)—Green group, similar to 139A.
   Leaf color (under side)—Green group, similar to 147B.
   Leaf arrangement.—Alternate.
   Leaf shape.—Elliptic.
   Leaf margins.—Entire.
   Leaf venation.—Reticulate.
   Leaf length.—Short, average 52.4 mm.
   Leaf width.—Broad, average 34.6 mm.
   Leaf length/width ratio.—1.5.
   Leaf nectarines.—Absent.
   Pubescence of upper side.—Absent.
   Pubescence of lower side.—Absent.
   Cross sectional profile.—Flat.
   Longitudinal profile.—Straight.
   Attitude.—Horizontal.
Petioles:
   Length.—Average 4.4 mm.
   Width.—Average 1.45 mm.
   Color.—Yellow green group, similar to 145B.
Flowers:
General:
   Time beginning of flowering.—Late season.
   Flower shape.—Urceolate.
   Flower fragrance.—None perceptible.
Corolla:
   Color.—White group, similar to NN155B.
   Length.—8.8 mm.
   Width.—8.9 mm.
   Aperture width.—4.8 mm.
   Anthocyanin coloration of corolla.—Absent or very weak.
   Corolla ridges.—Present.
   Protrusion of stigma.—Present.
Inflorescence:
   Surface texture of peduncle.—Medium.
   Color of peduncle.—Yellow-green group, similar to 145B with greyed-red group 182A on top.
   Length of pedicel.—10.8 mm.
   Surface texture of pedicel.—Smooth.
   Color of pedicel.—Yellow green group, similar to 146C.
   Number of flowers per cluster.—8.
   Flower cluster density.—Dense.
Calyx (with sepals):
   Diameter.—6.44 mm.
Stamen:
   Length.—7.6 mm.
   Number per flower.—10.
   Filament color.—Yellow-green group, similar to 145D.
   Style.—Length 10.5 mm.
   Color.—Yellow-green group, similar to N144B.
Pistil:
   Length.—12.3 mm.
Anther:
   Length.—4.4 mm.
   Number.—10.
   Color.—Greyed-orange group, similar to 165B.
Pollen:
   Self compatibility.—Yes.
   Abundance.—Abundant.
   Color.—Yellow group, similar to 10A.
Fruit:
General:
   Time of fruit ripening.—Late season.
   Cluster density.—Dense, range of 6-9 berries per cluster.
   Unripe fruit color.—Yellow green group, similar to 144B.
   Ripe Berry color.—Blue group 103A.
   Berry surface wax abundance.—Medium.
   Berry weight.—Large berry, on average 3 g.
   Berry height from calyx to scar.—13.6 mm.
   Berry diameter.—18.9 mm.
   Berry shape.—Oblate.
   Fruit stem scar.—Large.
   Sweetness when ripe.—Medium to high (13 Brix).
   Firmness when ripe.—Every firm.
   Acidity when ripe.—Medium to low (0.4%).
   Storage quality.—Low, similar to that of the bench mark variety ‘Snowchaser’ and average of 17 days.
   Suitability for mechanical harvesting.—Not tested.
   Self-fruitfulness.—Yes, with a proportion of fruit parthenocarpic.
   Uses.—Fruit to be hand harvested for fresh market.
Seed:
General:
   Seed abundance in fruit.—Low, on average 9 seeds per fruit.
   Seed color.—Greyed orange group, similar to 165A.
   Seed length.—2.16 mm.

### COMPARISON BETWEEN PARENTAL AND COMMERCIAL CULTIVARS

Table 1 below provide comparison between ‘C15-270’ and several commercial cultivars:

TABLE 1
Comparison of ‘C15-270’ with similar cultivars

| Characteristics | ‘C15-270’ | ‘C99-042’ | ‘Snowchaser’ |
|---|---|---|---|
| Plant vigour | Strong | Week to | Medium |
| | | medium | |
| Plant growth habit | Semi upright | Semi-upright to | Semi-upright |
| | | intermediate | |
| One year old shoot | Medium | Very short to | Short |
| length of internodes | | short | |
| Leaf length | Short | Short | Long |
| Leaf length (mm) | 52.4 ± 1.5 | 53.10 ± 0.8 | 61.50 ± 0.9 |
| Leaf width | Broad | Very narrow to | Broad |
| | | narrow | |
| Leaf width (mm) | 34.6 ± 1.4 | 22.9 ± 1.3 | 34.3 ± 1.5 |
| Flower size of corolla | Small to | Medium | Medium |
| | medium | | |
| Flower corolla | 8.8 ± 0.3 | 9.7 ± 0.3 | 9.5 ± 0.3 |
| length (mm) | | | |
| Fruit cluster density | Dense | Sparse | Medium |
| Fruit size | Large | Small to | Small to |
| | | medium | medium |
| Fruit weight (g) | 3 ± 0.3 | 1.9 ± 0.39 | 1.7 ± 0.21 |
| Fruit diameter (mm) | 18.9 ± 1.3 | 15.6 ± 0.96 | 15.10 ± 0.97 |
| Fruit intensity of bloom | Medium | Weak to | Weak to |
| | | medium | medium |
| Fruit firmness | Very firm | Firm | Soft |
| Soluble solid content (%) | 13 | 13.1 | 14.3 |
| Titratable acidity (%) | 0.4 | 0.3 | 0.5 |
| Time of vegetative | Late | Early | Early |
| bud burst | | | |
| Time of beginning | Late | Early to | Very early to |
| of flowering | | medium | early |
| Time of beginning | Late | Early to | Very early to |
| of fruit ripening | | medium | early |

1. A new and distinct variety of blueberry plant named ‘C15-270’, substantially as illustrated and described herein.
